(19) United States
(12) Patent Application Publication
(10) Pub. No.: US 2026/0306021 A1
(43) Pub. Date: Oct. 8, 2026

(54) Interchangeable Watch Strap with Removably Secured Electronic Sensing Module

(71) Applicant: Malcolm Conley, Fairfield, CT (US)

(72) Inventor: Malcolm Conley, Fairfield, CT (US)

(21) Appl. No.: 19/346,713

(22) Filed: Oct. 1, 2025

Related U.S. Application Data
(60) Provisional application No. 63/782,706, filed on Apr. 3, 2025.

(51) Int. Cl. A61B 5/00; A61B 5/024; A61B 5/1455; H02J 50/10; H02J 50/90

(52) U.S. Cl. CPC A61B 5/681; A61B 5/0022; A61B 5/02427; A61B 5/14552; A61B 5/7455; H02J 50/10; H02J 50/90; A61B 2560/0214; A61B 2560/0443; A61B 2560/0475; A61B 2562/0219; A61B 2562/08

(57) ABSTRACT

A modular watch strap system configured to enable biometric monitoring functionality for traditional wristwatches is disclosed. The system includes a strap body with an integrated frame proximate to the buckle, the frame defining a cavity adapted to removably receive a biometric sensor module. The sensor module comprises one or more physiological sensors, a power source, and a wireless communication interface for transmitting health data to a mobile device. The sensor maintains skin contact through the strap's underside when worn, enabling the collection of biometric data such as heart rate and blood oxygen levels. The removable module may be transferred between compatible straps of varying styles, materials, and widths, providing functional integration without altering the timepiece itself. Charging is facilitated via magnetic or inductive interfaces. The invention offers smartwatch-like health tracking capabilities while preserving the aesthetic and functional qualities of analog or mechanical watches.

### BACKGROUND OF THE INVENTION

### Field of Invention

The present invention relates generally to wearable electronic devices and more specifically to watch straps configured for health and biometric monitoring. More particularly, the invention pertains to an interchangeable wristwatch strap incorporating a removably secured sensor module that is adapted to collect physiological data from the wearer. The invention resides at the intersection of traditional horology and wearable health technology, and is especially relevant to systems enabling biometric functionality in analog or mechanical wristwatches without requiring permanent modification of the watch case.

### BRIEF SUMMARY OF THE INVENTION

The present invention is directed to a modular watch strap system configured to enable biometric monitoring functionality in conjunction with traditional wristwatches, including mechanical and quartz timepieces. The invention comprises a watch strap incorporating an integrated frame or receptacle positioned proximate to the strap buckle. This frame is configured to securely receive and retain a removable sensor module adapted to contact the inner wrist of the wearer during use. The sensor module includes one or more biometric sensors, such as heart rate, blood oxygen level (SpO₂), and similar physiological monitoring components, and is communicatively coupled to a mobile device via a wireless communication protocol such as Bluetooth®.

The frame is compatible with multiple strap formats and dimensions, allowing for interchangeability of the sensor module among a wide array of watch strap styles, materials, and widths. The sensor module is removably secured within the frame by way of a mechanism e.g., Snap-fit (detent-based locking), magnetic retention, screw-in fastening, bayonet mount, slide-lock mechanism, latch-and-hook, twist-lock interface, elastic or deformable retention tabs, adhesive backing (temporary/reusable), mechanical clip or clamp, rail and groove system, spring-loaded pin engagement, overmolded elastomeric gasket, cam-lock, rotary latch, etc. and/or a retention lip, and may be easily detached for charging, replacement, or transfer between straps. When worn, the module and frame are concealed from view, preserving the traditional aesthetic of the wristwatch. The invention thus enables wearers of non-smart watches to benefit from health tracking features commonly associated with smartwatches, without compromising design, comfort, or interchangeability of watch straps.

### BRIEF DESCRIPTION OF THE FIGURES

FIG. 1 is a side, front, and back profile view illustrating the sensor frame and the corresponding removable smart sensor module.

FIG. 2 is a cross-sectional view of the strap body showing the integration of the sensor frame and the sensor module housing.

FIG. 3 is a perspective view showing the sensor module in both an installed and a removed state relative to the sensor frame.

FIG. 4 is a CAD-style top-down view of the watch strap and sensor frame, illustrating spatial orientation and component layout.

FIG. 5 is a CAD-style side profile view of the strap assembly and sensor frame, depicting height, depth, and integration.

FIG. 6 is an exploded rendering of the invention showing each component prior to assembly.

FIG. 7 is a fully assembled rendering of the watch strap system, shown without strap loops.

FIG. 8 is a fully assembled rendering of the watch strap system, shown with strap loops included.

FIG. 9 is a perspective view of the watch strap showing the integrated biometric sensor module installed within the frame and including labeled components such as the keeper loop, frame upper, housing, and band body.

FIG. 10 is a perspective view of the complete watch strap assembly including the biometric sensor module and an installed keeper loop for securing the strap tail.

FIG. 11 is a perspective view of the strap and biometric module assembly without the keeper loop, showing the exposed frame and sensor module installed.

FIG. 12 is an orthographic view of the sensor module housing with key dimensional measurements labeled, illustrating the size and geometry of the biometric module.

FIG. 13 is a perspective view of the combined lower and upper frame elements used to retain the biometric sensor module, with visible positioning bumps used for secure engagement.

### DETAILED DESCRIPTION

The present disclosure relates to a watch strap assembly comprising an interchangeable wristband and a removable biometric sensor module. More particularly, the invention is directed toward a strap configured to interface with traditional analog, mechanical, or quartz timepieces, the strap being provided with an integrated retention frame for securing a health-monitoring sensor module that maintains contact with the wearer's skin during ordinary use. The disclosed system enables biometric data acquisition—typically afforded by dedicated smartwatches—while preserving the visual, tactile, and functional characteristics of conventional wristwatches.

In one embodiment, the invention comprises a two-piece watch strap constructed of flexible material suitable for wrist-worn applications, such as leather, silicone, thermoplastic elastomer (TPE), nylon, rubber, polyurethane, or combinations thereof. The strap is formed to interface with standard watch lug assemblies via conventional spring bars or quick-release pins and is therefore compatible with a wide variety of commercially available timepieces. The invention contemplates strap widths in a plurality of standardized dimensions, including, but not limited to, 18 mm, 20 mm, and 22 mm, thereby maximizing backward compatibility and user customization.

Embedded within one strap segment—preferably in the buckle segment—is a rigid or semi-rigid frame structure. Said frame defines a recessed cavity or housing aperture that traverses the depth of the strap and is dimensioned to receive a complementary electronic sensor module. The frame may be fabricated from polymeric materials such as acrylonitrile butadiene styrene (ABS), polycarbonate (PC), glass-filled nylon, or lightweight metallic materials such as anodized aluminum or stainless steel, with dimensional tolerances suitable for press-fit or interference-fit coupling with the sensor module. In preferred embodiments, the frame is co-molded or overmolded within the strap during manufacture, thereby ensuring structural integrity and alignment.

The cavity defined by the frame includes an open lower face directed toward the wearer's wrist, thereby exposing the sensor module's biometric interface when installed. The cavity may include a retention lip or overhang disposed along the cavity perimeter, such that the sensor module is retained within the frame when inserted from the underside of the strap. The lip extends partially into the cavity volume and may be continuous or segmented, defining a mechanical interference boundary that engages a corresponding groove, taper, or detent on the sensor housing. The sensor module is thus retained by friction, snap-fit, or a combination of mechanical interlocks, optionally facilitated by a designated push-out aperture or tool interface on the top surface of the strap.

The biometric sensor module comprises an encapsulated electronic assembly including one or more sensor elements selected from the group consisting of photoplethysmographic (PPG) sensors for heart rate detection, pulse oximetry sensors for blood oxygen saturation (SpO₂), electrodermal activity (EDA) sensors, skin temperature sensors, and accelerometers for motion analysis. The sensor package is configured such that the sensing interface, typically including optical emitters and detectors, is exposed through the lower face of the module and aligned with the cavity aperture such that uninterrupted contact with the wearer's skin is maintained during use. The sensor interface may include a transparent or semi-transparent window fabricated from a biocompatible polymer or glass, facilitating transmission of light for optical sensing while protecting internal components from mechanical or environmental damage.

The module housing further encloses a power source, typically a rechargeable lithium-polymer battery, and electronic circuitry necessary for sensor operation, data processing, wireless communication, and power management. The circuitry may include one or more microcontrollers, analog front-end signal processors, memory, and Bluetooth® transceivers. The module is operable to acquire, preprocess, and transmit physiological data wirelessly to a paired mobile device, such as a smartphone or smartwatch, wherein data is logged, interpreted, and/or displayed via a proprietary application or integrated platform such as Apple HealthKit™, Google Fit™, or Samsung Health™. The module may include onboard memory for temporary data storage in the absence of an active connection and may be programmed to transmit data asynchronously or in real time depending on use case and user preference.

The sensor module is further configured to be removable and rechargeable. Charging may be effected via magnetic pogo-pin connectors exposed on an upper or lateral surface of the module housing, or via inductive wireless charging coils embedded within the housing and aligned to interface with a compatible charging pad or dock. In embodiments employing magnetic charging contacts, the sensor may be charged using a bespoke charging clip or puck that magnetically aligns with the sensor module and establishes electrical contact through gold-plated pins. In embodiments employing wireless inductive charging (e.g., Qi-standard), no external contacts are required, thereby enhancing waterproofing and wear resistance.

The invention is particularly distinguished by its modularity and decoupling of the health-sensing functions from the timekeeping apparatus. Whereas existing solutions embed sensors within the caseback of the watch or permanently into the strap material, thereby compromising removability, serviceability, or aesthetic compatibility, the present invention permits full interchangeability of straps and modules without interfering with the functionality of the host watch. A single biometric module may be employed across multiple strap styles, materials, and sizes, provided the standardized frame geometry is maintained. In addition, the removable design allows the user to replace, upgrade, or remove the sensor module altogether without necessitating replacement of the watch strap or timepiece.

From a manufacturing perspective, the frame component may be molded as an independent sub-assembly and subsequently integrated into the strap via ultrasonic welding, adhesive bonding, mechanical stitching, or insert molding. Alternatively, the frame may be overmolded within the strap during a two-shot molding process, resulting in a monolithic strap with integrated sensor receptacle. In either case, the positioning of the frame within the strap is critical to the invention's function. In a preferred embodiment, the frame is located such that, when the strap is secured around the wrist, the sensor module lies in direct contact with the volar aspect of the wrist, near the radial artery, thereby optimizing the quality of biometric signal acquisition.

The aesthetic consideration is also a principal feature of the invention. When the strap is worn, the sensor module and its enclosing frame are positioned on the underside of the wrist and concealed from view. This allows the user to wear a traditional analog or mechanical wristwatch without visible indication of digital or smart components. The result is a device that preserves the heritage styling and tactile appeal of classic horological products while affording the user access to modern health and fitness monitoring capabilities.

The invention may be embodied in a variety of strap styles, including but not limited to: two-piece straps with buckle and tang; military-style pass-through straps; Zulu straps; deployant clasp-equipped straps; hook-and-loop fastener straps; or integrated lugs designs. Each strap embodiment may incorporate the standard frame structure and accommodate the same sensor module geometry, provided dimensional compatibility is maintained. Further, strap materials may vary depending on intended application—for instance, silicone or rubber for sport and fitness use, leather or suede for formal applications, or nylon for casual or utilitarian purposes.

In some embodiments, the sensor module may further include haptic components (e.g., vibration motors) for providing alerts, feedback, or notifications to the user. In such embodiments, the module may be programmed to emit haptic signals in response to physiological anomalies, fitness goals, connection status, or other user-defined events. Integration of haptic functionality may also enable silent alarms, breathing guidance, or posture correction feedback.

The system may optionally include anti-tamper or pairing mechanisms that associate a particular sensor module with a specific user profile or mobile application instance. Such mechanisms may include software-based authentication routines, near-field communication (NFC) pairing, or unique hardware identifiers. These mechanisms enhance data integrity, privacy, and usability, particularly in multi-user households or clinical settings.

In further embodiments, the sensor module may be constructed to meet ingress protection standards such as IP67 or IP68, thereby ensuring resistance to water, sweat, dust, and other environmental factors commonly encountered during daily use. The housing may be ultrasonically welded or sealed with gaskets to achieve such protection levels.

Referring to the anticipated figures, the invention may be further illustrated through diagrammatic and exploded views. A typical drawing set would include: (1) an isometric view of the strap and frame, (2) an exploded view showing the frame and sensor module prior to insertion, (3) a cross-sectional view illustrating the position and retention of the sensor module within the frame, and (4) illustrative views of the sensor module alone, highlighting key components such as the sensor interface, charging contacts, and housing geometry. Reference numerals may be used throughout the figures to identify corresponding structural elements such as the strap body, frame perimeter, retention lip, cavity, sensor module, sensor window, charging interface, and related components.

It is to be understood that variations in geometry, dimensions, materials, and feature positioning are within the scope of the present invention. For example, while a rectangular sensor module and corresponding frame are illustrated in some embodiments, alternative geometries such as elliptical, circular, or oblong housings may be employed. Similarly, while the frame is embedded in one strap segment in the preferred embodiment, it may be located elsewhere on the strap, and in some embodiments it desirable that skin contact and concealment is maintained.

Although the foregoing description has emphasized use with traditional watches, the invention may also be employed with smartwatch systems to extend health monitoring capabilities, act as secondary sensors, or provide redundancy. Furthermore, commercial deployment may involve integration with third-party platforms, cloud-based analytics, and health monitoring systems, particularly in fitness, wellness, or clinical trial contexts.

In some embodiments, the strap system may include user-guided interfaces, QR codes, or embedded RFID/NFC tags to facilitate app pairing, warranty validation, or inventory tracking. Such features may be implemented at the time of manufacture and tied to a backend server system that enables remote monitoring, software updates, or data aggregation.

Additionally, the invention is particularly suited for scalable manufacturing due to the decoupling of the sensor module from the strap body. A single module SKU (stock keeping unit) may be utilized across a broad inventory of strap styles, thereby simplifying logistics, reducing cost, and enhancing customer choice. Retailers or users may purchase multiple straps to match attire or activity without duplicating the cost of electronic components.

While preferred embodiments have been described in detail herein, it should be appreciated that various modifications, substitutions, and alterations may be made without departing from the spirit and scope of the invention as defined by the appended claims. Accordingly, the present disclosure is intended to be illustrative and not limiting in nature.

### DETAILED DESCRIPTION OF FIGURES

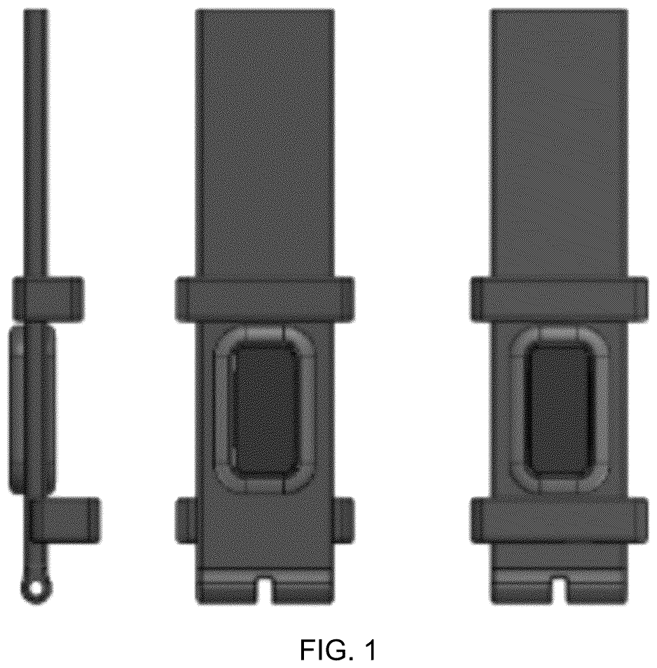

FIG. 1 illustrates three orthogonal profile views—side, front, and rear—of the sensor frame and the biometric sensor module, collectively forming the removable sensing assembly integrated within the watch strap. The side profile demonstrates the overall height of the sensor module in relation to the frame cavity and the tapering geometry of the module housing for friction-fit retention. The front view depicts the width of the module, showing alignment features such as notches or detents that interface with structural elements of the frame. The rear profile reveals the base portion of the module, which includes the biometric sensor interface, typically comprising optical components such as LEDs and photodetectors, protected by a transparent window for skin contact. The frame is shown with a rectangular or oval aperture and surrounding retention lip that mechanically secures the sensor module. The module's housing is shaped to slide into the frame cavity from below and is designed to seat flush against the strap body during normal use.

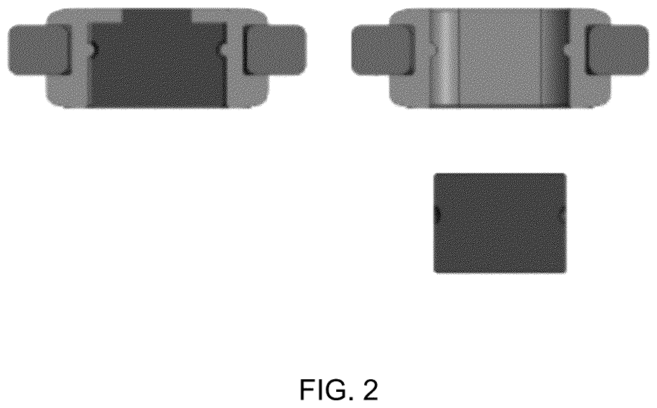

FIG. 2 provides a vertical cross-sectional view through the strap body, showing the integration of the frame and the press-fitted biometric sensor module. The strap body is represented as a layered structure composed of a flexible upper body and an embedded rigid or semi-rigid frame cavity. The cavity is positioned near the buckle and opens downward toward the wearer's wrist. The frame includes an inward-facing retention lip positioned approximately mid-depth along the inner walls of the cavity, designed to engage corresponding ridges or shoulders on the sensor module housing. The sensor module is shown nested within the cavity, its underside oriented toward the skin. Within the module, internal elements such as a rechargeable battery, a wireless communication chip, and optical sensing hardware are schematically represented. The strap body above the sensor is contoured to maintain comfort, while the module is flush with the bottom surface, ensuring continuous skin contact for optimal biometric acquisition.

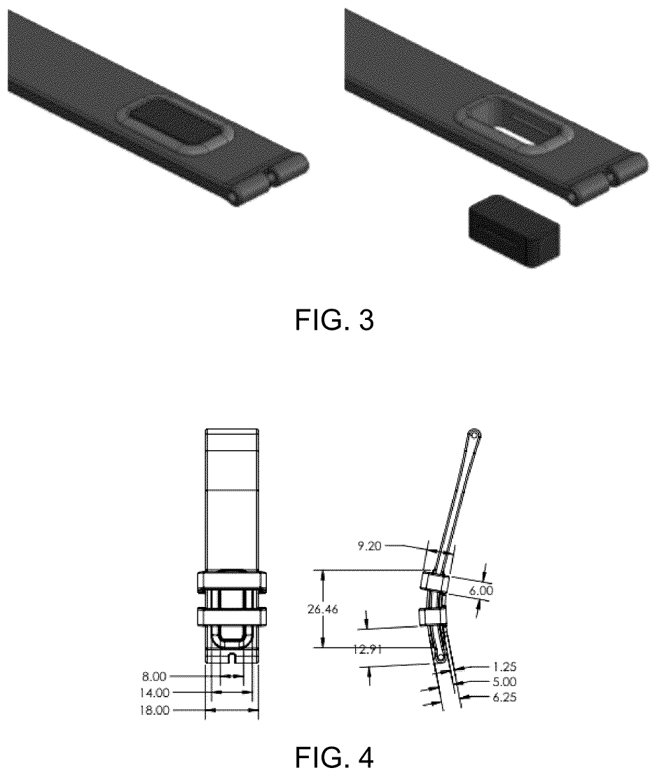

FIG. 3 illustrates the removable nature of the sensor module relative to the frame embedded in the strap. The figure includes two views: one showing the sensor module installed in the frame cavity, flush with the strap's bottom surface; and the other showing the module fully removed, aligned below the cavity opening to indicate its insertion path. The frame is embedded within the strap body, and the module housing is shaped to match the cavity geometry, facilitating guided alignment during insertion. The view may further include directional arrows or annotations indicating the direction of insertion and removal. Visible are the retention features—such as the frame's lip and the housing's engagement shoulder—that create a secure friction or snap-fit interface. The removed view also reveals the underside of the module, exposing the sensor window and, optionally, charging contacts or a wireless coil. This figure demonstrates the interchangeable functionality of the system and the non-permanent attachment of the sensor module to the strap.

FIG. 4 presents a computer-aided design (CAD) top-down view of the watch strap incorporating the sensor frame. This orthographic projection illustrates the general layout of the strap geometry, the relative position of the integrated sensor frame near the buckle end, and the shape of the frame cavity. The frame appears as a rectangular or elliptical recessed opening positioned centrally along the longitudinal axis of the strap. The strap includes standardized attachment ends compatible with spring bars or quick-release mechanisms, facilitating interoperability with a variety of wristwatch cases. From this view, alignment features, mounting surfaces, and internal boundaries of the sensor cavity may also be visible as part of the frame's internal geometry. The top surface of the strap is uninterrupted, maintaining aesthetic continuity and ensuring the biometric module remains hidden from view when installed.

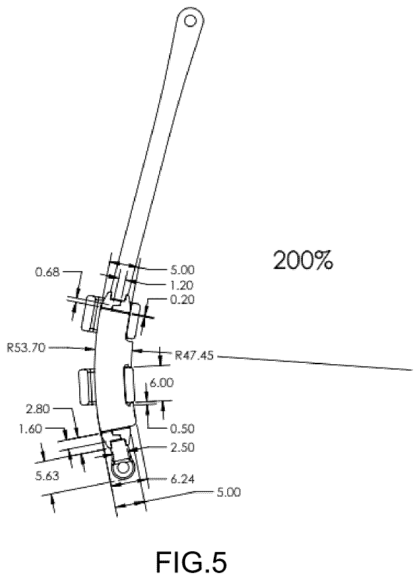

FIG. 5 provides a side profile CAD rendering of the strap and sensor frame assembly, focusing on the vertical relationship between the strap body, frame, and biometric sensor module. The figure highlights the integration of the frame within the thickness of the strap, showing how the sensor module sits flush with the bottom surface to maintain direct contact with the skin. The frame cavity is visibly embedded mid-way through the strap's thickness, with a downward-facing opening. This view further demonstrates the ergonomic curvature of the strap body, designed to conform to the natural shape of the wrist. The relative height of the sensor module, the contour of the strap above the module, and the compression-fit retention mechanism may also be visualized from this angle. The side profile allows for an appreciation of the low-profile construction and the minimal bulk added by the integrated sensing hardware.

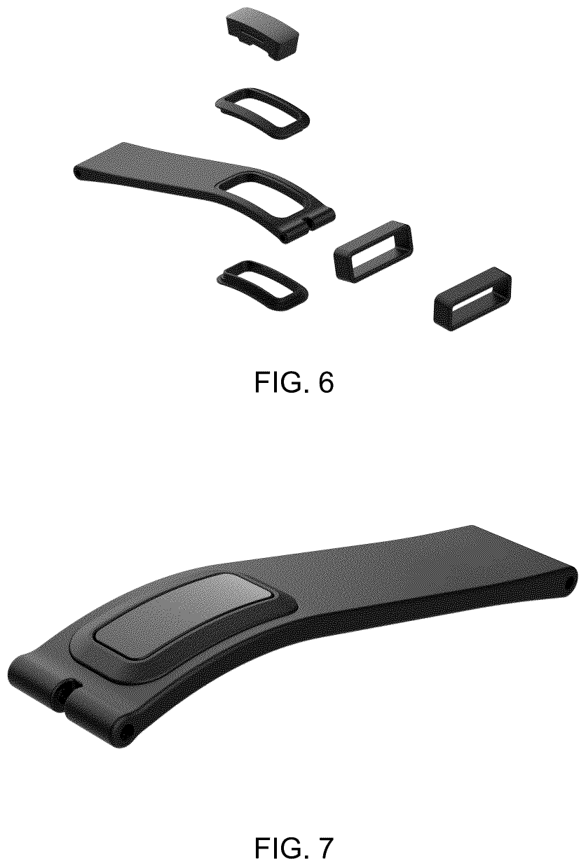

FIG. 6 is a digital rendering in exploded view, illustrating each discrete component of the invention prior to assembly. The components include, from top to bottom or front to back: the strap body (split into buckle and tail segments, if applicable), the integrated sensor frame (shown as a rigid insert or overmolded part), and the biometric sensor module, which includes its housing, sensor interface, and internal electronics. Optional components such as retention lips, cushioning elements, or structural ribs may also be shown. Arrows or spacing may be used to indicate assembly order and orientation. The exploded rendering highlights the modular nature of the invention, showing how the sensor module is manufactured separately from the strap and installed as a discrete unit. This figure also demonstrates how different materials and fabrication techniques contribute to the overall structure and functionality of the final assembly.

FIG. 7 is a digital rendering of the fully assembled strap system, shown without strap loops or keepers for visual clarity. The biometric sensor module is installed into the frame cavity and is visible only from the bottom-facing side of the strap. The buckle end, tail end, and integrated frame are all shown in a complete and wearable configuration. The absence of loops provides an unobstructed view of the strap's primary structural features and the seamless integration of the sensor housing into the strap body. The rendering may use realistic materials and lighting to illustrate finish options such as matte silicone, tanned leather, or woven nylon. This view demonstrates how the system appears during actual use, highlighting the discreet and aesthetically compatible nature of the smart module when paired with traditional wristwatches.

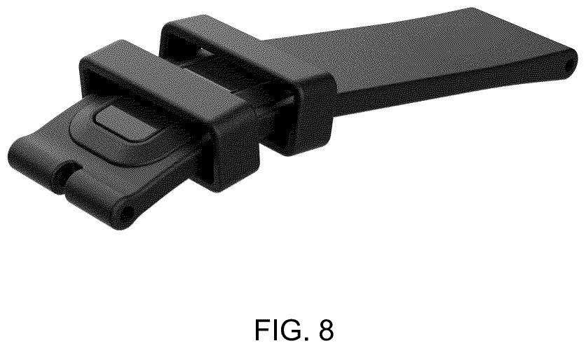

FIG. 8 depicts the fully assembled invention, including strap loops or keepers used to manage the excess tail portion of the strap once fastened. The loops may be fixed or sliding, and are shown in position relative to the sensor module and buckle assembly. The biometric sensor remains positioned on the underside of the strap, obscured from view when worn. This figure demonstrates the invention in its final, user-ready configuration, providing a realistic representation of the device during actual wear. The inclusion of strap loops illustrates compatibility with conventional watch strap ergonomics and shows how the added biometric functionality does not interfere with standard user interaction, wearability, or adjustment mechanisms. This figure also reinforces the continuity of the strap design, regardless of the added health-tracking features.

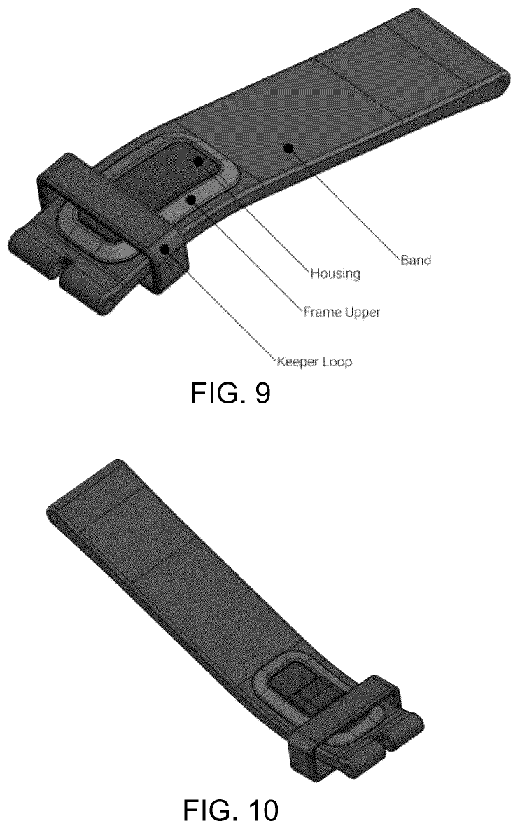

FIG. 9 illustrates a perspective view of the modular watch strap assembly, highlighting the integration of the biometric sensor module within the strap and the interaction of associated structural elements. The strap includes a flexible band portion terminating in a spring bar-compatible end, with an embedded frame upper forming a recessed cavity into which the sensor housing is installed. The housing is shown as flush or slightly inset into the cavity, maintaining alignment with the interior-facing surface of the strap for optimal skin contact. A keeper loop is depicted near the buckle region, used to secure excess strap length after fastening. The frame is embedded beneath the band's top surface and includes geometries to retain the housing in position via friction-fit or mechanical engagement. The figure demonstrates the clean integration of sensing functionality without interrupting the aesthetic flow of the traditional watch strap.

FIG. 10 presents a perspective rendering of the assembled strap in a configuration where the keeper loop is installed and visible on the upper side of the strap. The sensor housing is shown in its fully inserted state, flush with the inner curvature of the strap for ergonomic skin contact. The keeper loop, typically made of a flexible or semi-rigid material, is shown positioned to receive and secure the excess length of the strap tail once the strap has been passed through the buckle. This figure visually confirms that the addition of the biometric module and associated frame does not interfere with the standard operation of the watch strap, including common features such as loop retention. The depiction reinforces the interchangeable and unobtrusive design of the invention, highlighting its compatibility with conventional watch-wearing practices.

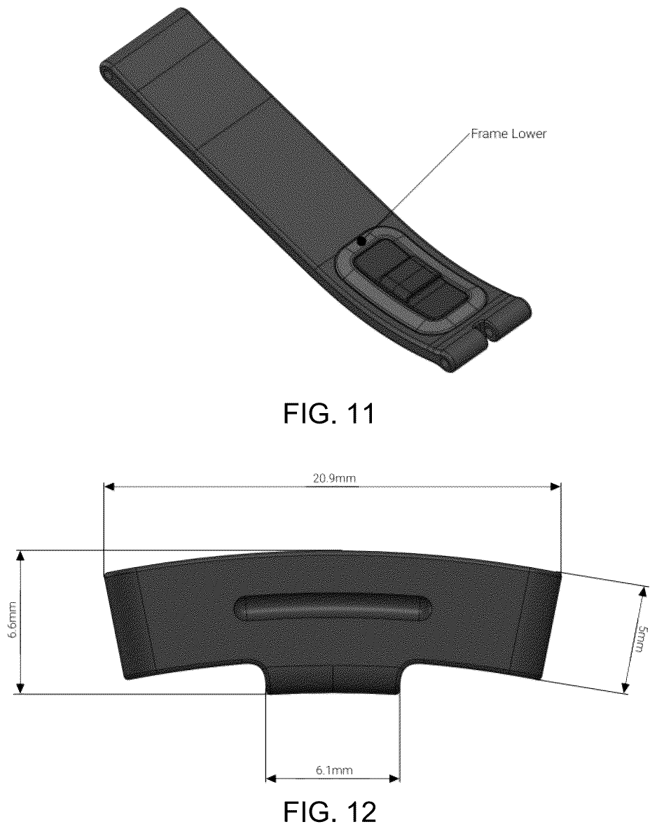

FIG. 11 shows the strap assembly in a configuration where the keeper loop has been removed or omitted, providing an unobstructed view of the integrated frame lower and the installed sensor module. The absence of the loop allows clear visibility of how the housing is retained within the frame embedded in the strap body. The figure emphasizes the curvature and ergonomic alignment of the strap body and sensor module, ensuring intimate skin contact while maintaining flexibility for wrist movement. The streamlined design demonstrates the modularity of the invention—highlighting that the strap may be worn with or without a keeper loop depending on user preference or design requirements—without compromising biometric functionality.

FIG. 12 provides a top-down orthographic view of the sensor housing, with critical dimensional parameters annotated. The overall width of the housing is labeled as 20.9 mm, with a height of 6.5 mm at its tallest point and 5 mm at its tapered end. The central channel or recess on the surface of the housing may correspond to a tactile or alignment feature for insertion into the frame. The 6.1 mm internal cutout dimension represents a narrowing or recess potentially used for secure fitting within the frame or for accommodating internal sensor components. This figure defines the spatial envelope and ergonomics of the sensor housing, confirming its compact, wrist-conforming design intended for seamless integration with a variety of strap sizes. The controlled taper ensures a press-fit engagement while maintaining comfort and consistent sensor-to-skin contact.

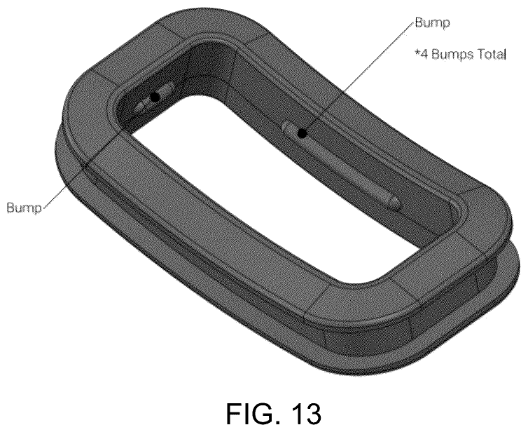

FIG. 13 depicts the frame assembly that houses the biometric sensor module, shown in a combined view of the frame lower and frame upper segments. The frame defines a central cavity into which the sensor module is inserted from below. Highlighted in this figure are four evenly spaced bumps located along the inner surface of the frame walls, designated as alignment or retention bumps. These raised features provide mechanical interference that engages complementary features on the sensor housing, ensuring a secure friction-fit and preventing unintentional dislodgment during wear. The inner cavity is dimensioned to match the external contour of the sensor housing shown in FIG. 12, providing both axial and radial alignment. The figure emphasizes the mechanical robustness and simplicity of the retention method, which avoids complex latching or clamping mechanisms while still enabling tool-free installation and removal of the sensor module.

What is claimed is:

1. A modular watch strap assembly for use with a wristwatch, the assembly comprising:
(a) a strap body configured to attach to a watch case via spring bar connectors, the strap body having a top surface and a bottom surface;
(b) a frame integrated into the strap body at a location proximal to a buckle or fastening element, the frame defining a cavity having an open bottom surface configured to face a wrist of a wearer when the strap is worn;
(c) a electronic sensor module removably secured within the cavity of the frame, the sensor module comprising:
(i) one or more physiological sensors configured to detect biometric data from the wearer via the open bottom surface of the cavity;
(ii) a housing dimensioned to frictionally engage with the frame;
(iii) a wireless communication interface configured to transmit biometric data to a mobile computing device; and
(iv) a power source disposed within the housing and configured to supply electrical power to the sensor module;
wherein the sensor module is removably insertable into and ejectable from the cavity of the strap body, and wherein the frame and sensor module are configured such that the sensor module maintains skin contact during wear.

2. The modular watch strap assembly of claim 1, wherein the biometric sensor module comprises a photoplethysmographic sensor configured to detect heart rate data.

3. The modular watch strap assembly of claim 1, wherein the biometric sensor module comprises a pulse oximetry sensor configured to detect blood oxygen saturation (SpO₂) levels.

4. The modular watch strap assembly of claim 1, wherein the biometric sensor module comprises a wireless charging interface including a coil adapted to receive inductive energy from an external charging pad.

5. The modular watch strap assembly of claim 1, wherein the biometric sensor module comprises a magnetic charging interface including electrical contacts and a magnet for alignment with a corresponding charging apparatus.

6. The modular watch strap assembly of claim 1, wherein the housing of the sensor module includes a transparent window formed of biocompatible material, the window positioned to permit optical sensing of the wearer's skin.

7. The modular watch strap assembly of claim 1, wherein the frame includes a retention lip circumferentially or partially encircling the cavity, the lip configured to mechanically engage the housing of the sensor module and resist removal during normal use.

8. The modular watch strap assembly of claim 1, wherein the strap body is formed from a material selected from the group consisting of cotton, polyester, leather, silicone, rubber, thermoplastic elastomer (TPE), nylon, or polyurethane.

9. The modular watch strap assembly of claim 1, wherein the sensor module is operably coupled to transmit biometric data to a mobile device via a Bluetooth® protocol.

10. The modular watch strap assembly of claim 1, wherein the strap body comprises a one-piece or two-piece configuration including a buckle strap and a tail strap, and wherein the frame is embedded in the buckle strap.

11. The modular watch strap assembly of claim 1, wherein the biometric sensor module further comprises an accelerometer configured to detect motion or activity of the wearer.

12. The modular watch strap assembly of claim 1, wherein the biometric sensor module is configured to interface with a proprietary health application or a third-party platform.

13. The modular watch strap assembly of claim 1, wherein the frame is permanently affixed to the strap body via overmolding during a multi-shot injection molding process.

14. The modular watch strap assembly of claim 1, wherein the frame is mechanically or adhesively bonded to the strap body post-manufacture.

15. The modular watch strap assembly of claim 1, wherein the sensor module includes onboard memory configured to store biometric data prior to wireless transmission.

16. The modular watch strap assembly of claim 1, wherein the frame and the biometric sensor module are configured to conform to at least one of a plurality of strap widths, including 18 mm, 20 mm, or 22 mm.

17. The modular watch strap assembly of claim 1, wherein the sensor module includes haptic feedback components configured to deliver tactile alerts to the wearer.

18. The modular watch strap assembly of claim 1, wherein the biometric sensor module is configured to operate in a low-power mode when not actively transmitting data.

19. The modular watch strap assembly of claim 1, wherein the housing of the biometric sensor module includes a unique identifier for use in device authentication or user pairing.

20. The modular watch strap assembly of claim 1, wherein the strap body includes a push-out aperture or deformable region on the top surface of the strap for facilitating manual removal of the sensor module.
